(19) United States
(12) Patent Application Publication
(10) Pub. No.: US 2015/0343708 A1
(43) Pub. Date: Dec. 3, 2015

(54) MEDICAL 3D PRINTING CONEX

(71) Applicant: OSIRIS BIOMED 3D, LLC, Valhalla, NY (US)

(72) Inventors: Theodore L. GERSTLE, Valhalla, NY (US); Christopher GERSTLE, Valhalla, NY (US)

(21) Appl. No.: 14/723,421

(22) Filed: May 27, 2015

Related U.S. Application Data
(60) Provisional application No. 62/003,477, filed on May 27, 2014.
(60) Provisional application No. 62/076,384, filed on Nov. 6, 2014.

(51) Int. Cl. B29C 67/00; G05B 15/02

(52) U.S. Cl. CPC B29C 67/0088; G05B 15/02; A61B 2017/00526

(57) ABSTRACT

The present disclosure provides devices and methods for obtaining images of body parts, implants, instruments and models to provide for a mobile hospital, operating room, and/or facility to print desired tools or prostheses. A Conex of the present disclosure includes a computer, image scanning device, printer, and raw materials, and optionally an autoclave and second, product verification scanner. All of these components will be co-located within the Conex. The operator of the Conex will also have access to a database with stored data relating to the large number of objects needed in surgical applications (such as tools and anatomical norms of bone and tissue), allowing the ability to field a mobile hospital at a greatly reduced weight, cost and time.

### CROSS-REFERENCE TO RELATED APPLICATIONS

The present application claims the benefit of U.S. Provisional Application Ser. No. 62/003,477, filed on May 27, 2014, and U.S. Provisional Application Ser. No. 62/076,384, filed on Nov. 6, 2014, each of which is incorporated herein by reference.

### BACKGROUND OF THE DISCLOSURE

1. Field of the Disclosure

The present disclosure relates to methods and devices for scanning and 3D printing. More particularly, the present disclosure relates to a Conex trailer that can include a three-dimensional printing lab.

2. Description of the Related Art

In surgical operations, there is a need to have a wide variety of instruments and prostheses available to treat patients. Exigency can be high in some applications. For example, when treating wounded military personnel, the wounds are often severe and need to be addressed as soon as possible. In addition, in both military and non-military applications, medical practitioners may perform surgeries that require the use of tools and/or a replacement prosthesis. These practitioners may be limited by space, or how much and what types of equipment can be carried into the field. The present disclosure addresses these deficiencies.

### SUMMARY OF THE DISCLOSURE

The present disclosure provides an apparatus and process for producing tools and/or prostheses for surgical procedures. The disclosure provides a Conex with three-dimensional (3D) printing capability that would replace the need for sending or stocking large quantities of surgical instruments and customized prosthetics. This can decrease the shipping costs to the surgical site, and the time to create a prosthetic implant or tool for the surgery. The Conex can contain a scanner, computer, 3D printers and raw materials for printing surgical instruments or prostheses. The present disclosure thus overcomes the need for shipping and/or storing large quantities of medical devices, instruments and prostheses at a medical facility when they could be quickly printed on-site. The present devices and methods will allow for mobile surgery centers to be constructed quickly with only a need for a Conex.

The methods and devices of the present disclosure will allow for direct virtual private network (VPN) image sharing so that doctors in the field can receive images and collaborate with other physicians to assist them in their access to the proper implants/prosthesis and troubleshoot designs for a specific surgery. This is highly advantageous, as multiple difficult trauma cases often present themselves in challenging locations (e.g., on a battlefield or theater of military operation). The devices and methods of the present disclosure help to provide the highest standard of care to the patient.

The devices and methods of the present disclosure can also have access to a database that stores information related to medical devices, instruments, and prostheses. They can also have the ability to make customized implants on site with a scanning and printing method and create life-saving and therapeutic devices that are able to help patients in time of need. Through all of these features, the present disclosure can decrease shipping weight of necessary surgical equipment, decrease time to definitive implantation of life-saving medical implantable devices, and create operating rooms in a matter of hours. Raw materials can be supplied through prepackaged proprietary means and delivered through high-fidelity Federal Drug Administration (FDA) approved methods.

For ease of description, the terms “prosthesis” and “prostheses” are used in the present disclosure to refer to the types of implants, bone replacements, tissue replacements, prostheses, or even whole organs that can be designed and created in the devices and with the methods of the present disclosure. Thus, the term “prosthesis” as used in the present disclosure may refer to customized facial implants (bony or soft tissue implantation), facial fractures and repair, microtia framework, ocular prostheses, nasal prostheses, maxillary prostheses, palatal prostheses, septal prostheses, cranial vault prostheses, mandibular bone replacement (bone graft printout), maxillary bone replacement, customized soft tissue implant (all areas of the body including but not limited to airway stents, vascular stents, grafts, percutaneous or surgical vascular occlusion devices), hand/extremity implants/prostheses, joint replacement (e.g., small joints of the wrist/fingers), large joint replacement (e.g., hips, knees, shoulder), spine corpus replacement, long bone replacement (femur, tibia, fibula, radius, ulna, humerus), rib cage replacements, pelvic defect repairs, large joint replacements, non-implantable prosthetics (e.g., fingers, other appendages, limbs, orthotics, or obturators), combinations thereof, or other suitable implants.

Thus, in one embodiment, the present disclosure provides a process of producing tools and prosthetics by way of a mobile Conex printing lab. The method comprises the steps of: either accessing a database of stored data relating to a plurality of surgical instruments or prostheses or scanning a target to acquire an image of the surgical instrument or prosthesis; selecting an image relating to one of the plurality of surgical instruments or prostheses; displaying the image on a display device; sending the image to a printer; and printing the instrument or prosthesis according to the image on the printer.

In another embodiment, the present disclosure provides a Conex for use in mobile surgical applications, comprising: a computer; a three-dimensional printer; and raw materials, wherein the printer uses the raw materials to print a surgical instrument and/or a prosthesis.

### BRIEF DESCRIPTION OF THE DRAWINGS

FIG. 1 shows a flowchart describing how a user can upload designs to the database for sharing using several different imaging or mapping programs for data input.

FIG. 2 shows an example of a Conex that can be used in the present disclosure.

### DETAILED DESCRIPTION OF THE DISCLOSURE

The Conex of the present disclosure can contain a computer, a 3D printer or printers, and raw materials for printing surgical instruments or prostheses. The Conex may also include, optionally, an image acquisition device, an autoclave or other device for sterilizing the outputs of the 3D printer, and a scanner to verify the printed product(s). The present disclosure also provides methods for using the same. An image of the desired surgical instrument or prosthesis is obtained with an image acquisition device within the Conex, an image acquisition device remote to the Conex, or by accessing a database with stored image data relating to the same. The image is then sent to the printer for printing. In this way, the present disclosure provides devices and methods for ultra-rapid prototyping of prosthetics/tools for surgical applications. One suitable application for the Conex of the present disclosure is in the military, as it allows for the creation of mobile military medical facilities within hours. The Conex can be prepositioned prior to its need. Other applications and features are described in greater detail below.

The devices and methods of the present disclosure are discussed in the context of three-dimensional (3D) printing (also known as “additive manufacturing”). 3D printing may include, but is not limited to, such methods as fused deposition modeling, fused filament fabrication, robocasting, electron beam freeform fabrication, direct metal laser sintering, electron beam melting, selective laser melting, selective heat sintering, selective laser sintering, plaster-based 3D printing, laminated object manufacturing, stereolithography, and digital light processing. Processes of “subtractive” manufacturing may be employed as well. In this embodiment, the image acquisition device would send an image of a desired prosthesis to the computer, as described above. The final image, with or without modification, is sent to a fabricator. The fabricator uses subtractive methods to produce the prosthesis, where the prosthesis can be hewn from a solid piece of implantable material. The subtractive methods may include lathing the prosthesis, cutting with laser-, water-, or air-blade-cutting tools, stamping, grinding, or carving.

By “intra-operative use”, the present disclosure means that the prosthesis and/or instrument is printed or fabricated within the same operative procedure (i.e., under a “single aesthetic”) or in the same operative location as the location where the image on which the prosthesis is based is acquired. Currently available devices or methods may refer to “rapid-prototyping”, but this typically means that when the image of a specific part is acquired, it is then sent off to be printed remotely, in a process that may take several weeks. With use of the terms “ultra-rapid prototyping” and “intra-operative”, the present disclosure distinguishes over these processes. In the method of the present disclosure, the required prosthesis can be provided during the surgical procedure. One of the most unique aspects of this disclosure is that the scanning of the patient and processing of the image as well as printing of the prosthetic or other implantable devices for the patient can be done under a single anesthetic.

The term “Conex” is used in the present disclosure for convenience, and denotes an enclosure or container suitable for modular, containerized, intermodal transport. Any enclosure suitable for storing the components of the present disclosure, allowing for use as a mobile fabrication room or an operating room, and providing the ease of transport described herein is suitable. Examples of Conex boxes are shown in the following, which is herein incorporated by reference: http://en.wikipedia.org/wiki/Intermodal_container.

According to the present disclosure, a user, doctor, or technician can acquire a three-dimensional image of a prosthesis/instrument. The image can be acquired with an image acquisition device. This image acquisition device can be a camera, haptic mapping or imaging device, a magnetic resonance imager (MRI), x-ray device, computerized axial tomography (CAT) scanner, or similar device, which can be in communication with a computer. The data that the scanner acquires or sends to the computer can include any information relevant to the instrument or prosthesis in question. The data can include dimensions, shapes, tolerances, and other specifications relating to the instrument or prosthesis. The image may also be acquired via a database stored on the computer within the Conex, or residing on a remote server or cloud service. The database can store information relating to existing designs of prosthesis or tools. As discussed in greater detail below, the database of the present disclosure can also store normative data relating to prostheses or tools.

A software program or algorithm can be embedded on the computer, and can cause the acquired image to be shown on a monitor or other display. The software program can allow the doctor, a technician, with or without input from the patient themselves, to customize the scanned image to desired settings or features. The final image, customized if applicable, is then sent to the printer or fabricator for creation. As previously discussed, the printer or fabricator is within the Conex, with the computer. This drastically reduces the amount of time required to produce the prosthesis used in the surgical procedure.

In one embodiment, the image acquisition device, computer, and printer or fabricator are co-located within the Conex. In another embodiment, the computer, printer, sterilization device, and verification scanner can be co-located within the Conex. Either way, the devices and methods of the present disclosure are located so that ultra-rapid prototyping is possible, eliminating or significantly reducing the amount of delay in obtaining a required prosthesis or instrument. The period of time that the printer or fabricator provides the prosthesis after obtaining the final image can vary, depending on the particular type of medical procedure. This period of time can range from ten minutes to twenty-four hours, or any sub-ranges therebetween. The period of time can also be from thirty minutes to twelve hours, or any subranges therebetween.

When the Conex of the present disclosure is used in surgical applications, the actual surgical procedure on the patient can take place within the Conex. The present disclosure also contemplates that the surgical procedure can take place outside of the Conex, so that the Conex serves as a fabrication room for the prosthesis or surgical tool produced. The Conex can be used to print the desired tool or prosthesis, and package it for delivery to another facility or location. The packaging can be done in a sterilized manner if desired. As a non-limiting example, the Conex of the present disclosure can be deployed on the grounds of an existing hospital facility. A user can print a desired tool or prosthesis, and the Conex can package it in a sterilized package. The user can then transport the packaged part to the site where the surgical procedure is to take place.

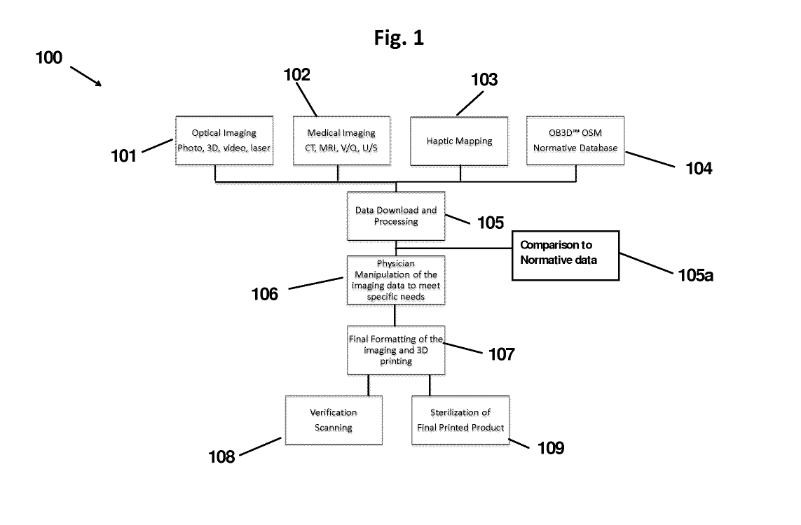

Referring to FIG. 1, a process (100) according to the present disclosure is shown. First, a user may acquire an image with an optical scanning device (101), a medical imaging device (102), haptic or touch mapping (103), or through the afore-mentioned database of stored instrument or anatomical data (104). The image is then downloaded and processed by the computer (105). Optionally, the computer can display the image to the user or physician for further manipulation (106). After this step, the image is then sent off to the printer for printing (107). A verification scanner can optionally verify that the final printed product is correct (108). Also optionally, the final printed product can be sterilized with a sterilizing device, e.g. an autoclave (109).

There may also be an optional step (105a), between the downloading step (105), and displaying step (106). Due to imperfections in image acquisition technology, when a body part or instrument is scanned, there may be missing information, gaps, or “holes” in the final image. If this image were sent to the printer or fabricator, it would be imperfect and thus unsuitable. During step (105a), the image can be compared to normative data for the prosthesis or tool that can be stored in the database. Any gaps in the acquired image can be filled in. This is known as creating a “water-tight” image.

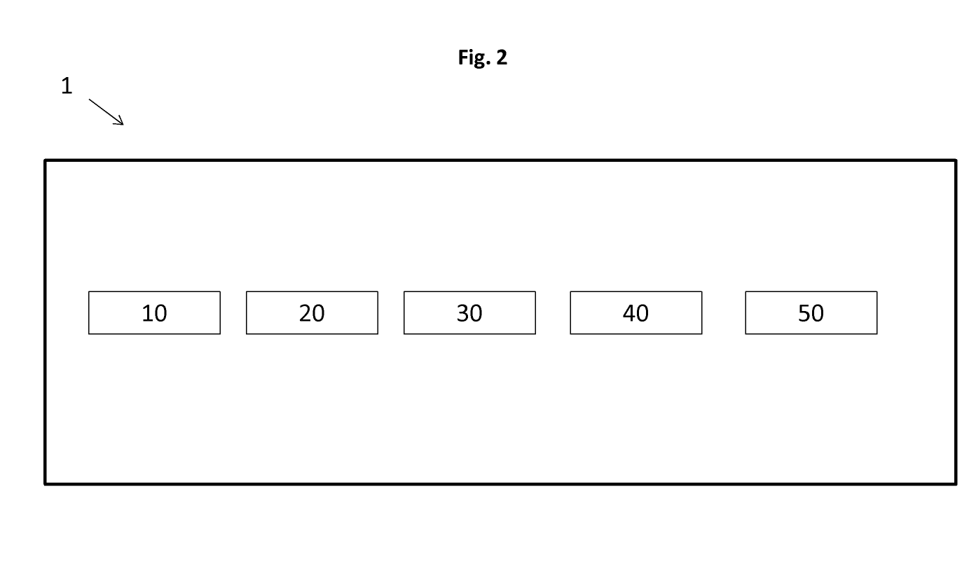

Referring to FIG. 2, a schematic view of the Conex of the present disclosure is shown, and referred to by numeral 1. Conex 1, as described above, is a suitable container that can be mobile, and hold all of the required components described herein. Conex 1 can be, for example, a corrugated metal shipping container. In one embodiment, these shipping containers can have a length of from eight to fifty-six feet, or any subranges therebetween. The height can be from eight to nine and one half feet, or any subranges therebetween.

Conex 1 can optionally contain the image acquisition device 10. Conex 1 further contains computer 20 and printer 30. This is what the present disclosure means by having device 10, computer 20, and printer 30 co-located. Each of these components is within Conex 1, during use, shipping, and/or installation of Conex 1. When used, verification scanning device 40 and autoclave 50 can also be within Conex 1. Computer 20 can have algorithm 25 thereon, which performs the image acquisition and manipulation functions described above. Verification scanning device 40 scans the final, printed product, and confirms that the printed product conforms to the image that was sent to printer 30. Algorithm 25 can also perform this verification function.

With respect to military applications, soldiers are currently stabilized on the battlefield and evacuated to a secondary site (e.g., Germany) for further medical care. With the methods discussed in this disclosure, more definitive surgery could take place prior to evacuation of the soldier from a theater of operations which could mean saving their life, eyesight, or a limb. Using the Conex of the present disclosure and the database with stored data relating to surgical instruments and prostheses, the devices and methods of the present disclosure can produce a complete operating room with disposable instruments, which can be constructed quickly (<24 hours) and cheaply by the methods in this disclosure. Therefore, the only stockpile that would need to be provided to construct the instruments for the surgeon and anesthesiologist would be the designs from the database and the raw materials for printing. These could be used to construct anything from implants and scalpels to flexible or reinforced endotracheal tubes, tracheostomy tubes, or airway stents. Lastly, the cost of buying implants or prosthesis under currently available methods can be extremely high, often as much as $10,000. The devices and methods of the present disclosure, used for printing a patient specific implant, will save the military large sums of money as it will eliminate the need for large stockpiles of implantable prosthetics that can become non-sterile and cause a traumatic infection.

Other suitable applications for the Conex of the present disclosure could be in disaster areas. In the wake of a natural disaster such as a hurricane or earthquake, traditional or currently available power systems and medical services can be disrupted. The Conex of the present disclosure can be deployed in such situations for urgent onsite medical assistance. The Conex of the present disclosure can also be used in existing medical facilities (e.g. an urban hospital), where space may be of a premium and additional flexibility to perform surgeries that may or may not be part of the facility's standard capabilities. The use of the Conex in this latter application may also be beneficial to free up existing storage space in these facilities, which can be used to stockpile custom or non-custom implants.

The devices and methods of the present disclosure, including the database, can provide a means to change the way support hospitals are constructed. This will provide the needed cost savings and will make the needed devices and prosthetics available to a physician in the field to provide a better quality of care to a patient.

The printer or fabricator of the present disclosure can also eliminate the time associated with sterilization of an implantable prosthesis in currently available devices and methods. Currently, when a surgeon receives an implantable prosthesis after the printing delay, there is additional time associated with sterilization of the prosthesis, which further adds to the cost of the procedure and risk for the patient. With the devices and methods of the present disclosure, however, this time is significantly reduced or eliminated completely. The printer or fabricator provides with the devices and methods of the present disclosure can provide an already-sterilized prosthesis for immediate use. In the case of a prosthesis produced via computer-guided lathe, the machining of the prosthesis will still likely still require sterilization, but the lathing process can be more expeditious than printing, so the additional time for sterilization should not be significant.

The materials suitable for the prostheses of the present disclosure may vary. The materials can include polylactic acid and acrylonitrile butadiene styrene, which are approved by the United States Food and Drug Administration for implantable devices. Other materials contemplated may include rubber, light-cured polymers, metals, ceramics, and implantable antibiotic-impregnated solids.

In addition to being suitable for implanting prostheses in patients, the devices and methods of the present disclosure can provide surgical planning models and cutting guides for the doctor and patient. The doctor can hold a model of a bone or skull, for example, and develop a plan of where incisions or bone removal are to take place. The doctor can also illustrate the same to the patient or the patient's caregiver or guardian.

As discussed above, although the Conex or other suitable modular container of the present disclosure can be used for three-dimensional printing in military applications, other applications are contemplated. The Conex of the present disclose may be used in any application where its mobility is useful to provide convenient medical and/or laboratory services, in a variety of locations. For example, in natural disaster areas where power infrastructure and access to equipment may be disrupted, where medical centers have been compromised, or in other remote areas that are not necessarily in theaters of war. The three-dimensional printing aspect of the present disclosure is particularly beneficial, but the Conex of the present disclosure could also contain other medical equipment, tools, or prostheses that are pre-fabricated or printed.

The Conex of the present disclosure can also be used in applications where mobility is not a primary concern. For example, an existing hospital facility may need the ability to print tools or prostheses rapidly, and may not currently have the ability to do so, or have the space. The Conex of the present disclosure can be easily deployed on the grounds of an existing hospital facility, for example in a parking lot, parking garage, rooftop, or unused area of the facility's grounds. The Conex of the present disclosure can also be deployed wherever its capabilities are needed, whether there was an existing facility or not.

While the present disclosure has been described with reference to one or more particular embodiments, it will be understood by those skilled in the art that various changes may be made and equivalents may be substituted for elements thereof without departing from the scope thereof. In addition, many modifications may be made to adapt a particular situation or material to the teachings of the disclosure without departing from the scope thereof. Therefore, it is intended that the disclosure not be limited to the particular embodiment(s) disclosed as the best mode contemplated for carrying out this disclosure.

1. A process of producing tools and prosthetics by way of a mobile Conex printing lab, comprising the steps of:
either accessing a database of stored data relating to a plurality of surgical instruments or prostheses or scanning a target to acquire an image of said surgical instrument or prosthesis;
selecting an image relating to one of said plurality of surgical instruments or prostheses;
displaying the image on a display device;
sending the image to a printer; and
printing the instrument or prosthesis according to said image on said printer.

2. The process of claim 1, wherein said method steps are performed during a single anesthetic, intra-operative procedure.

3. The process of claim 1, wherein said scanning device and said printer are co-located within the Conex.

4. The process of claim 1, wherein a period of time between said sending step and said printing step is between thirty minutes and twenty-four hours.

5. The process of claim 1, further comprising, after said displaying step and before said sending step, allowing a user to customize said image according to desired parameters.

6. The process of claim 1, further comprising, after said printing step, scanning said instrument or prosthesis to verify that it matches desired preset parameters.

7. The process of claim 1, further comprising, after said printing step, sterilizing said instrument or prosthesis.

8. The process of claim 1, further comprising, after said printing step, packaging said instrument or prosthesis.

9. A Conex for use in mobile surgical applications, comprising:
a computer;
a three-dimensional printer; and
raw materials,
wherein said printer uses said raw materials to print a surgical instrument and/or a prosthesis.

10. The Conex of claim 8, further comprising an image acquisition device.

11. The Conex of claim 10, wherein said image acquisition device, computer, printer, and raw materials are all co-located within the Conex.

12. The Conex of claim 9, further comprising a database in communication with said computer, wherein said database stores image data relating to said surgical instruments and/or prostheses.

13. The Conex of claim 12, wherein said computer communicates said image data to said printer, to print said surgical instruments and/or prostheses.

14. The Conex of claim 9, further comprising a verification scanner, to scan said surgical instrument and/or said prosthesis.

15. The Conex of claim 14, further comprising a database in communication with said computer, wherein said database stores image data relating to said surgical instruments and/or prostheses, wherein said computer communicates said image data to said printer, to print said surgical instruments and/or prostheses, and
wherein said verification scanner acquires an image of said surgical instrument and/or said prosthesis and compares said image to said image data communicated to said printer.

16. The Conex of claim 14, wherein said computer, said printer, said raw materials, and said verification scanner are all co-located within the Conex.

17. The Conex of claim 9, further comprising a sterilizing device, to sterilize said surgical instrument and/or said prosthesis.

18. The Conex of claim 17, wherein said computer, said printer, said raw materials, and said sterilizing device are all co-located within the Conex.
